(19) United States
(12) Patent Application Publication
(10) Pub. No.: US 2023/0404484 A1
(43) Pub. Date: Dec. 21, 2023

(54) WEARABLE DEVICE

(71) Applicant: J and M TECHNOLOGIES LTD, Bath (GB)

(72) Inventors: Gavin JENNINGS, Bath (GB); Peter MCGRATH, Gloucestershire (GB); Andrew BARR, Antrim (GB)

(21) Appl. No.: 18/035,728

(22) PCT Filed: Nov. 9, 2021
(86) PCT No.: PCT/EP2021/081170

§ 371 Date: May 5, 2023

(30) Foreign Application Priority Data
Nov. 9, 2020 (GB) 2017658.2
Jul. 8, 2021 (GB) 2109875.1

(51) Int. Cl. A61B 5/00; A61B 5/107; A61N 1/36

(52) U.S. Cl. CPC A61B 5/6831; A61B 5/1071; A61B 5/0022; A61N 1/36021; A61B 5/742

(57) ABSTRACT

A wearable sensor is described which provides real-time joint range of motion data. Also described is a sensor for providing real-time joint motion data to assess the capability of a joint. The sensor may comprise an inertial measurement unit and/or a combination of tri-axial accelerometer, tri-axial goniometer, and tri-axial magnetometer.

The present invention relates generally to a device which can be worn and can provide data relating to, and/or benefits for, a wearer.

Aspects and embodiments of the present invention may provide or relate to wearable devices delivering different (possibly distinct) benefits which may be used individually, separately or in combination.

One aspect provides a wearable sensor for providing real-time joint range of motion data.

Range of motion refers to the movement potential of a joint from full extension to full flexion (bending). Range of motion, also known as ROM, is a measure of flexibility involving ligaments, tendons, muscles, bones, and joints, so testing for ROM is essential in determining fitness and in assessing possible damage. Full range of motion indicates that the particular joint has the ability to move in all the directions it is supposed to move.

There are three basic types of range of motion: passive, active-assistive and active, defined by the whether, and to what degree, the patient can move the joint voluntarily. Passive ROM exercise is performed by a physical therapist or medical practitioner alone, active ROM is performed independently by the patient, and active-assistive ROM may involve the use of bands, straps or other devices, accompanied by verbal directions.

A further aspect provides a sensor for providing real-time joint motion data to assess the capability of a joint.

Sensors formed in accordance with aspects and embodiments of the present invention may comprise an IMU that comprises an accelerometer, gyroscope and a magnetometer to provide a position in space.

The sensor may, for example, comprise a combination of tri-axial accelerometer, tri-axial goniometer and tri-axial magnetometer.

The sensor may continuously report its orientation about its own internal tri-axial frame.

The sensor may comprise a magnetometer component which makes use of the earth's magnetic field in order to help determine the orientation of the sensor relative to the surface of the planet.

The sensor may be standalone (e.g. not needing to be used in conjunction with other such sensors).

The sensor may be used in combination with a brace, strap or the like.

The sensor may be used in combination with a pain relieving device such a neurostimulator.

The present invention also provides a method to provide real-time joint range of motion data comprising use of a sensor or combination as described herein positioned in one area of a subject to monitor that area.

The present invention also provides a method to provide real-time joint range of motion data comprising use of a sensor or combination as described herein to monitor a site distant/remote from the physical sensor location.

Methods may comprise the step of positioning one sensor at a known initial orientation and then subsequently reporting changes in angular orientation of the sensor relative to the initial orientation without reference to any other sensor.

The present invention also provides a wearable device comprising means for monitoring motion of or around a joint, in which the device incorporates means for providing pain relief and/or symptom alleviation.

The device may be provided as a strap, brace or the like.

The device may comprise a pain relieving neurostimulator means, such as TENS or PENS.

Precise locational data with associated software can, for example, allow for an exact replication of the joint movement to be shown to the patient ensuring complete compliance with the complexities of the rehabilitation schedule.

The patient is therefore better able to follow instructions which can be sent and updated in real time by the physician or healthcare professional

An application of this aspect may be to monitor patients recovering from joint or limb related surgery or limb injury and/or to check progress pre- to post-operatively.

Some embodiments are based on a principle of using a sensor positioned in one area of a subject to monitor that area. Alternatively or additionally the sensor may be used to monitor a site distant/remote from the physical sensor location. For example one or more sensors placed around the knee and elbow could be used to monitor plains of movement across the hip and shoulder.

Sensor hardware, in conjunction with the associated firmware, may allow for both hip and knee movements to be monitored with two, single or just a single (for example) device sensor/s housed in removable strapping/brace placed around the knee.

Both elbow and complex shoulder movements could, for example, be monitored with two, single device sensors, or just a single sensor, housed in removable strapping/brace worn around the elbow. Uniquely, this allows measurement of not only single plane motion such as hip, knee and shoulder flexion and extension, but also abduction/adduction movements and rotation movements which are of particular value in hip and shoulder rehabilitation.

The device may, for example, connect to a patient app on a mobile phone or tablet device which may show:
The patient in real time may see the degree of movement obtained with each repetition of exercise.
May also show averages and trends over course of rehabilitation.
Two-way feedback ability for both clinicians, patients and health providers to maintain contact.

The device may connect to clinician app on e.g. PC allowing remote monitoring of patient to avoid need for many face-to-face consultations and to inform clinician by way of an ‘alarm’ process if there were any concerning qualitative or qualitative deviations in terms of patient compliance and progress.

Sensor technology may incorporate the use of any of the components of Inertial Measurement Units or 3d Motion technology. This may allow for more detailed measurements to be taken.

A further aspect relates to a wearable device that incorporates means for providing pain relief and/or symptom alleviation, for example neurostimulation.

The pain-relief aspect may, for example, be combined with the monitoring aspect.

The wearable device may, for example, be or form part of a pain relieving neurostimulator device such as TENS or PENS.

Other pain relief/symptom alleviation mechanisms may, for example, include means for heating and/or cooling and/or massage means (for example kneading, rolling, tapping, gripping or shiatsu based) for an affected area.

Some embodiments aim to use a pain-relieving function prior to exercises to allow faster rehabilitation and reduce requirement for oral analgesia such as opiates.

In some embodiments the pain-relieving device can be monitored in conjunction with the sensors to check if the use of a pain device speeds recovery.

Pain relief could, for example be delivered via one or more pads placed at the point of pain or via a wearable device placed across the joint.

In some embodiments the aim is:
Monitoring sensors integrated into the wearable pain-relieving device. This would require sensors to work on all four joints where the pain device is used.
Used after the pain device with two pads have been used.

All associated software may, for example, present the same information with the following data:
Power level of pain relief delivered for each rehabilitation session to see how this relates to movement
Ability to check if rehab recovery times differ between the pads or wearable device.
Collection of data that can be used to show how (if) reducing pain with the device allows for not only quicker recovery but reduction in time pain relief is required.

In some aspects and embodiments the device is provided in the form of a brace, support, strap or the like with onboard sensors provided thereon or thereby.

In some embodiments the sensor/s used include an accelerometer and/or an inertial measurement unit (IMU), for example an IMU that comprises an accelerometer, gyroscope and a magnetometer to provide a precise position in space.

Types of joints which may be monitored by the present invention may include: i) simple joints (two articulation surfaces e.g. shoulder joint, hip joint); ii) compound joints (three or more articulation surfaces e.g. radiocarpal joint); iii) complex joints (two or more articulation surfaces and an articular disc or meniscus e.g. knee joint).

Data may be transmitted from the device.

Data may be transmitted continuously. Alternatively or additionally data may be transmitted periodically from the device. The transmission of data from the device may be automatic or controlled/triggered by user/clinician input.

Data can be transmitted from the data capture device using a short-range wireless communications protocol.

Data may be transmittable to a proxy for onward transmission.

Some embodiment relates to a wearable orthopaedic device delivering two distinct benefits which may be used individually or in combination.

The two functions (monitoring and pain relief) may be provided by separate wearable devices. Alternatively the two functions may be provided by the same device.

Some aspects of the present invention use or require a plurality of sensors or sensor modules. Some aspects and embodiments use or require only a single sensor or sensor module.

In some aspects and embodiments each sensor module is entirely stand-alone and does not act like a conventional goniometer which relies upon the difference signal between one side of a joint and the other in order to obtain an angle. Instead each sensor module may be continuously reporting its orientation about its own internal tri-axial frame.

A significant contribution to this absolute reporting is a magnetometer component of the sensor which makes use of the earth's magnetic field in order to help determine the orientation of the sensor relative to the surface of the planet.

Therefore, by positioning one sensor at a known initial orientation it is then possible subsequently to report all changes in angular orientation of this sensor relative to the initial orientation without reference to any other sensor.

Although only one sensor is required to measure multiplanar movement at a single joint, the use of two sensors either side of a joint, for example the knee, would, for example, allow monitoring of the entire limb segment simultaneously i.e. both hip and knee movement as well as movement of the femur and tibia relative to the pelvis could all be visualised simultaneously.

Uniquely for a single sensor, it is not absolutely necessary to align the axes of the sensor with the axes of rotation of the limb segment—any misalignment can be compensated for within a mathematical model. However, better alignment means that any errors in the mathematical model will make a correspondingly smaller impact on the final measurement of angular orientation.

If, therefore, one axis of the sensor is aligned parallel to the long axis of the limb segment then the sensor will report at all times rotation angle of the limb segment about its long axis.

For this reason, as the anatomical features on one side of a joint are stationery throughout the examination—such as at the opposite shoulder or hip joint, then it is only necessary to deploy a single sensor on the upper arm or on the thigh in order to obtain the angular displacement of the upper arm relative to the line through the shoulder-blades or to obtain the angular displacement of the thigh relative to the line through the pelvis.

As shown in the previous point, when only a single sensor is required for measuring angles at the knee, hip or shoulder and elbow the sensor is placed either at the upper arm for the shoulder and elbow or thigh for the knee and hip. The mathematical model associated with the sensor compensates for slight changes in the positioning of the sensor meaning it doesn't need to be placed in exactly the same place every time.

The sensor/s can, for example, be placed in a strapping around the arm or thigh (the nature of this strapping is not limited and could be decided upon at the point of use or the preference of the patient/clinician).

In some embodiments the sensor is a combination of tri-axial accelerometer, tri-axial goniometer and tri-axial magnetometer. It may employ firmware to interpret the raw signals from these constituent components in order to derive angular orientation data for the sensor about each of three mutually orthogonal axes. This orientation data may be determined relative to an arbitrary cartesian coordinate frame of reference so that at any time the orientation of the sensor may be defined as lying parallel to any particular axis of the frame of reference (and thereby having an angular offset of zero) such that all subsequent measurements of orientation are defined relative to that reference orientation. This is important because it means that the sensor may be fitted to a limb in any orientation, and the wearer may be instructed to adopt any reference posture for that limb, and the sensor may then be ‘zeroed’ to that postural position, such that all subsequent measurements are defined relative to that reference postural position for the limb.

The model that interprets the raw signals may be an algorithm. It may be supplied as an accessory to an IMD (inertial measurement device) or IMU (inertial measurement unit) component to support its use for converting raw signals into angular orientation.

The mathematical model for converting IMD output into meaningful limb and joint angles may be based on simple vector arithmetic.

Fundamentally the importance of the IMD based sensor of embodiments of the present invention is that it can report changes in orientation of a limb segment from a starting posture without reference to any other limb segment. This means that if a limb is moved relative to other non-moving anatomical parts, then the angle formed by that limb segment and the non-moving anatomical parts may be reported as a simple function of the single sensor. Specifically if the line between the shoulder blades has not moved and only the upper-arm has moved relative to that line between the shoulder blades—then it is sufficient to track the changes in orientation of the upper arm in order to report changes in angle between the upper arm and the line between the shoulder blades

Why measure?

The vast majority of patients who have undergone a musculo-skeletal elective operation require post-operative monitoring to ensure the appropriate milestones in rehabilitation are being achieved. The same applies to many patients who have sustained musculoskeletal trauma, even some of those who have been treated non-operatively.

Joint range of movement is a prime metric in determining the progress of rehabilitation. Monitoring of compliance with prescribed rehabilitation programmes is also of significant use. It is well documented in the medical literature that compliance improves when the patient is aware they are being monitored more closely.

The traditional method of monitoring rehabilitation progress is during specific ‘point prevalence’ assessment at face to face clinic appointments, either with the responsible surgical team or physical therapist. This requires moderate resource investment, time and travel for the clinician and/or the patient. Assessing progress at isolated time points does not allow trends to be monitored with the same level of granularity as can be achieved with continuous monitoring. Being able to identify trends in range of movement at earlier time points is beneficial in terms of identifying potential issues. For example, following joint replacement, loss of movement may suggest potential infection, which if identified earlier may allow much simpler treatment than if an infection becomes more established. Another example would be following many shoulder operations where a frozen shoulder is a fairly common complication. Early identification of frozen shoulder would alert the clinician to the need for urgent assessment and potential steroid injection. It is also well recognised that early injection is more likely to resolve the problem than if the injection is delayed.

What data will be collected?

The sensors will have the ability to measure range of movement across multiple joints either individually or as compound limb movements. Data is collected and transmitted in real time giving the patient a visual demonstration of the range achieved contemporaneously on a screen such as on a mobile phone. Data is also collected on the average and maximum ranges achieved within a session along with quantitative information such as the number of exercise sessions and repetitions of each exercise completed within each session.

Pain scores data and other PROMS can also be collected at relevant time points.

The data will be available both to the patient and also the clinician, who can monitor the progress and compliance remotely.

Over time, a data repository will be formed which will contain information for example, on patients' behaviours, pre-operative movement and post-operative gains with specific joint replacement protheses, the benefits of specific rehabilitation protocols etc.

Health Insurers would potentially find data useful both on the individual patient and collectively to confirm and promote compliance and demonstrate the optimal patient pathways to ensure the best outcome in the shortest time frames.

How will the data be displayed?

There will be two distinct portals displaying slightly different data.

The patient portal, for example, will replicate on an avatar, and also numerically in degrees, the movement of each individual repetition in real-time to allow the patient to confirm that they are achieving the minimum desired range with each exercise and to also confirm that they are not exceeding recommended upper limits if these have been stipulated. The patient portal can also have other helpful information embedded, such as video demonstrations of how to complete the exercises correctly. The portal will also show the patient their individualised rehab program which can be configured by the clinician. Pain scores can be entered by the patient at each session and other PROMS data entered as required.

There will also be a facility to message the clinician with any queries or concerns.

The clinician portal will list patients currently being monitored and will have an alert system if any of those patients vary from their expected progress in terms of range of movement trends, pain scores, compliance etc. The alert system will draw attention to any issues and avoid the need for the clinician to continually check each individual patient's progress. This will also allow the clinician to reduce the number of necessary face to face post-operative consultations and reserve clinic appointments to see (in a more timely fashion) those patients where issues have been identified through remote monitoring. The clinician can also send collective or individual notifications through the application.

Sensor Detail

The or each sensor module may be entirely stand-alone and does not act like a conventional goniometer which relies upon the difference signal between one side of a joint and the other in order to obtain an angle. Instead, each sensor module is continuously reporting its orientation about its own internal tri-axial frame.

A significant contribution to this absolute reporting is the magnetometer component of the sensor which makes use of the earth's magnetic field in order to help determine the orientation of the sensor relative to the surface of the planet.

Therefore, by positioning one sensor at a known initial orientation we can then subsequently report all changes in angular orientation of this sensor relative to the initial orientation without reference to any other sensor.

Although only one sensor is required to measure multiplanar movement at a single joint, the use of two sensors either side of a joint, for example the knee, would allow monitoring of the entire limb segment simultaneously e.g. both hip and knee movement, as well as movement of the femur and tibia relative to the pelvis, could all be visualised simultaneously.

Uniquely for a single sensor, it is not absolutely necessary to align the axes of the sensor with the axes of rotation of the limb segment—any misalignment can be compensated for within a mathematical model. However better alignment means that any errors in the mathematical model will make a correspondingly smaller impact on the final measurement of angular orientation.

If therefore one axis of the sensor is aligned parallel to the long axis of the limb segment then the sensor will report at all times the rotation angle of the limb segment about its long axis.

For this reason, as the anatomical features on one side of a joint are stationery throughout the examination—such as at the opposite shoulder or hip joint, then it is only necessary to deploy a single sensor on the upper arm or the thigh in order to obtain the angular displacement of the upper arm relative to the line through the shoulder-blades or to obtain the angular displacement of the thigh relative to the line through the pelvis.

As shown in the previous point, when only a single sensor is required for measuring angles at the knee, hip or shoulder and elbow the sensor is placed either at the upper arm for the shoulder and elbow or thigh for the knee and hip. The mathematical model associated with the sensor compensates for slight changes in the positioning of the sensor meaning it doesn't need to be placed in exactly the same place every time.

The sensors can either be placed in a strapping around the arm or thigh (the nature of this strapping isn't limited and can be decided upon at the point of use or the preference of the patient/clinician.

The use of a single sensor to monitor multi-planar movements is unique and of commercial benefit as the cost per case is consequently reduced. The ability to use identical sensors and strapping for measurement of all the major joints is also unique.

Clinical Applications

The sensors will be used both in post-injury and post-operative care in hospital and physiotherapy clinic environments.

The sensor can be commissioned, patient instructions given and pre-operative movement determined at a pre-admission visit. The sensor can be applied to the relevant body segment with a strap or brace.

Multiplanar shoulder movements (flexion/extension, internal and external rotation and abduction/adduction) can be monitored with a single sensor attached around the upper arm. Elbow movements can be monitored with sensors either side of the elbow (with the same configuration able to monitor elbow, forearm, humeral and shoulder movements synchronously).

Multiplanar hip movements (flexion/extension, abduction/adduction and internal/external rotation) can be monitored with a single sensor strapped to the thigh.

Knee movements can be measured with a sensor either side of the joint (with the same configuration able to monitor tibia, knee, femoral and hip joint movement synchronously).

Pre-habilitation can be commenced if desired.

Postoperatively, monitoring can be commenced at the desired time point.

Different aspects and embodiments of the invention may be used separately or together.

Further particular and preferred aspects of the present invention are set out in the accompanying independent and dependent claims. Features of the dependent claims may be combined with the features of the independent claims as appropriate, and in combination other than those explicitly set out in the claims.

The present invention will now be more particularly described, with reference to the accompanying drawings.

All orientational terms, such as upper, lower, radially and axially, are used in relation to the drawings and should not be interpreted as limiting on the invention or its connection to a closure. Example embodiments are described in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed and as well as individual embodiments the invention is intended to cover combinations of those embodiments as well. On the contrary, all modifications, equivalents, and alternatives falling within the scope of the appended claims should be included. Elements of the example embodiments are consistently denoted by the same reference numerals throughout the drawings and detailed description where appropriate.

The terminology used herein is not intended to limit the scope. The articles “a,” “an,” and “the” are singular in that they have a single referent; however, the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealised or overly formal sense unless expressly so defined herein.

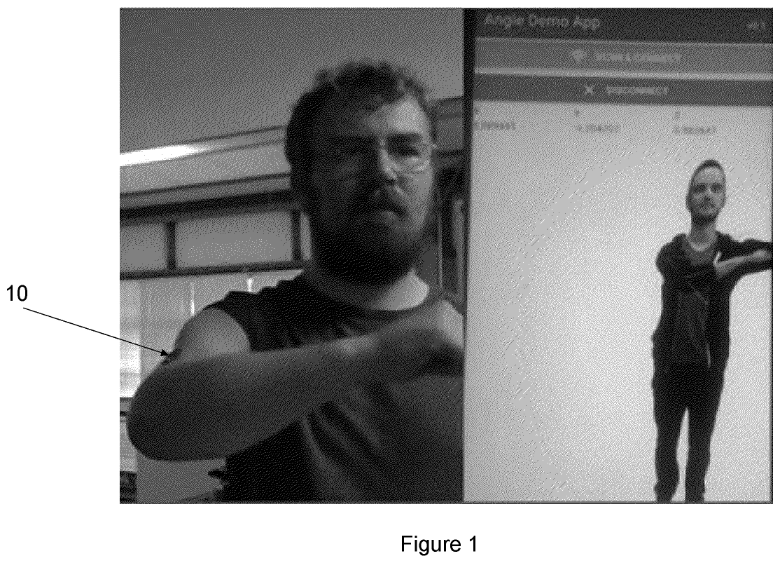

Referring first to FIG. 1 there is shown, on the left hand side, an individual wearing a sensor 10 along their arm (in this embodiment in between the shoulder and the elbow). On the right had side a representation of the position of the individual's arm is shown, based on data provided by the sensor 10.

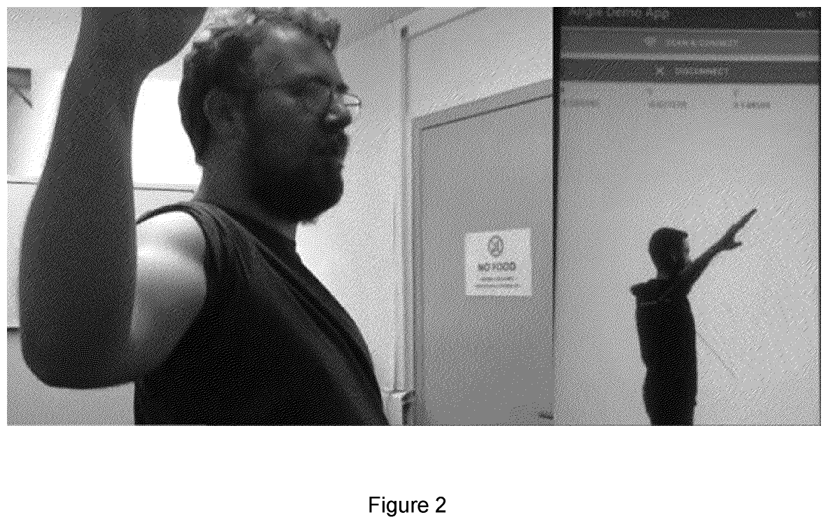

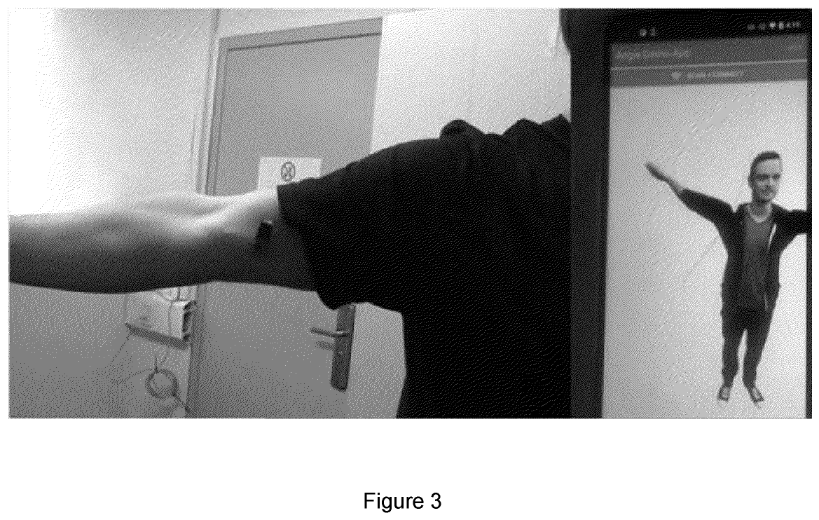

FIGS. 2 and 3 show different arm movements and sensor positions.

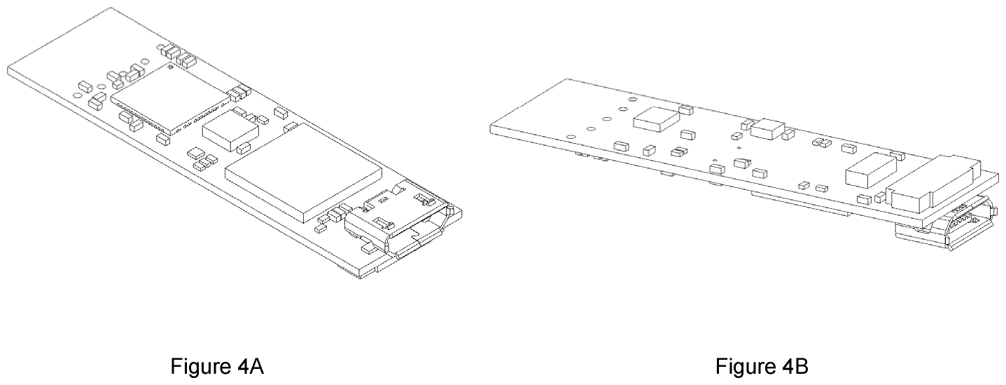

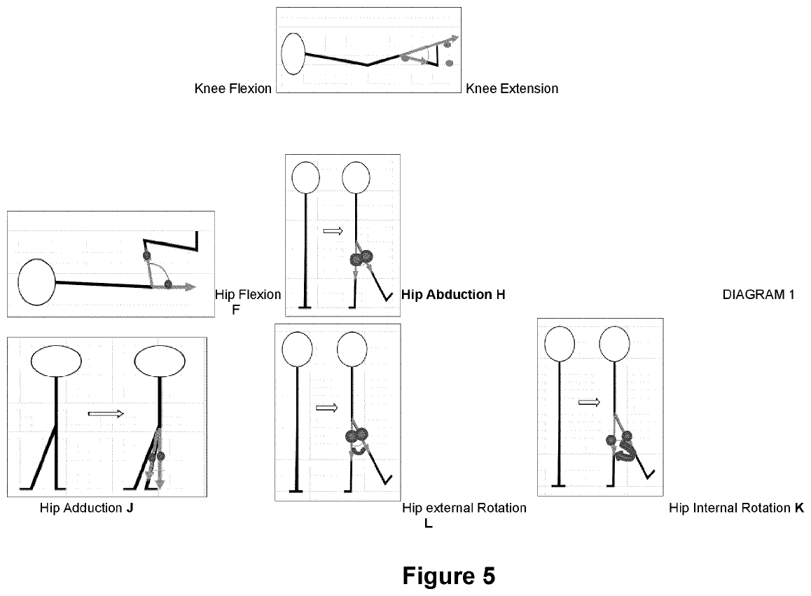

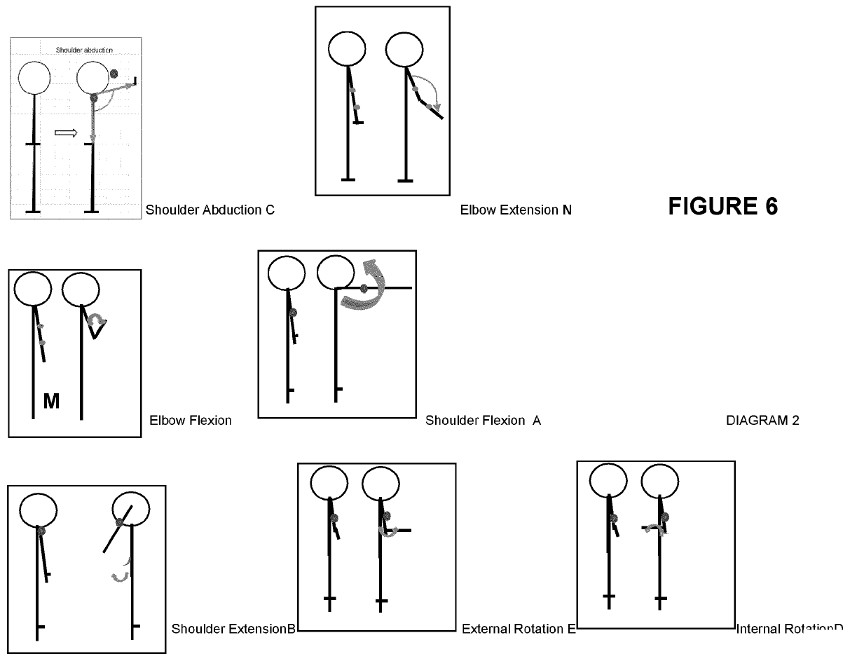

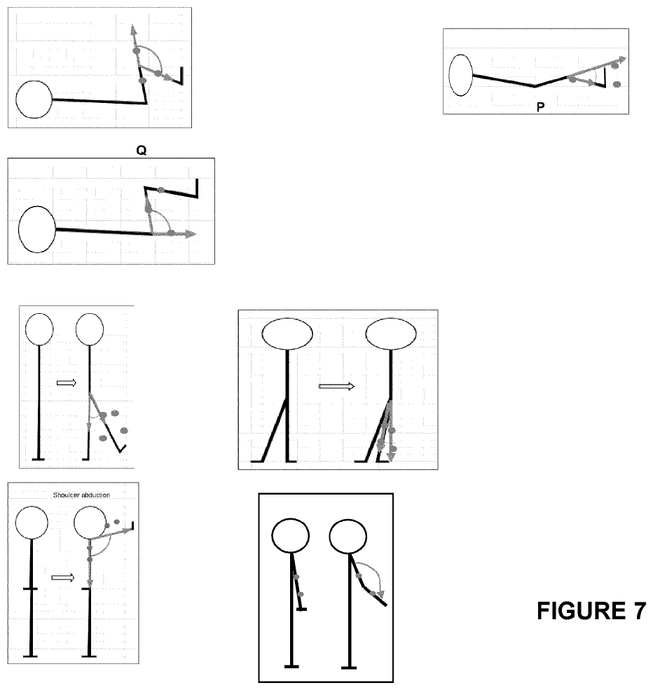

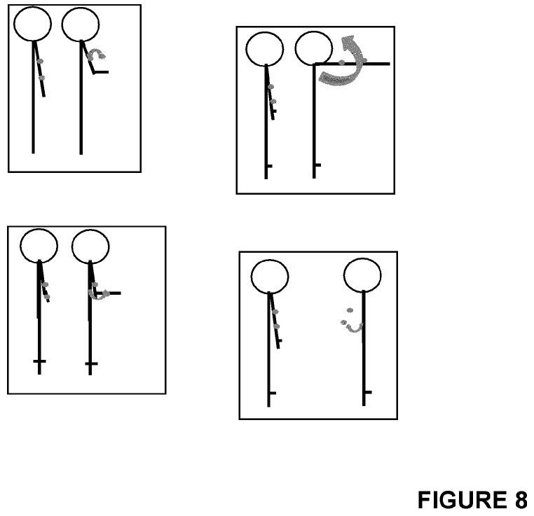

FIGS. 4A and 4B and are top side and bottom side line drawings of a printed circuit board (PCB) forming part of the sensor 10.

### WEARABLE SENSORS TO PROVIDE REAL TIME JOINT RANGE OF MOTION DATA

Main application would be to monitor patients recovering from joint or limb related surgery or limb injury.

Only sensors placed around the knee and elbow would be required to monitor plains of movement across the hip and shoulder e.g. Diagram 1.

Algorithms/sensor hardware, in conjunction with the associated firmware will allow for:
Both hip and knee movements to be monitored with two, single device sensors housed in removable strapping/brace placed around the knee.
Both elbow and complex shoulder movements could be monitored with two, single device sensors housed in removable strapping/brace worn around the elbow.

This allows measurement of not only single plane motion such as hip, knee and shoulder flexion and extension but also abduction/adduction movements and rotation movements which are of particular value in hip and shoulder rehabilitation e.g. Diagram 2.

Device would connect to a patient app on a mobile phone or tablet device which would show the following:
The patient in real time will see the degree of movement obtained with each repetition of exercise.
Would also show averages and trends over course of rehabilitation.
Two-way feedback ability for both clinicians, patients and health providers to maintain contact.

Device may connect to clinician app on e.g. PC allowing remote monitoring of patient to avoid need for many face to face consultations and to inform clinician by way of an ‘alarm’ process if there were any concerning qualitative or qualitative deviations in terms of patient compliance and progress.

Development of sensor technology could also incorporate the use of any of the components of Inertial Measurement Units or 3d Motion technology. This could be used in the same way as Diagram 1 and/or 2 but the data captured is far more accurate. Allowing for much more detailed measurements to be taken.

Also shown in the drawings:

Single Sensor

A single sensor can monitor the following:
Upper limb:
Mulitplanar shoulder movements including:
Flexion and extension (A, B)
Abduction (C)
Internal and external rotation (D, E)
Lower limb:
Multiplanar hip movements including:
Flexion and extension (F)
Abduction/adduction (H, J)
Internal and external rotation (K, L)

Two Sensors

Use of two sensors can allow monitoring of:
Upper Limb:
Wrist flexion/Extension
Elbow flexion and extension (M, N)
Whole limb segment movement i.e. forearm, elbow, upper arm and shoulder movements synchronously
Lower Limb:
Ankle flexion/extension
Knee flexion/extension (P, Q)
Whole limb segment movement i.e. lower leg, knee, thigh and hip movement synchronously

Wearable Pain-Relieving Device

Some aspects and embodiments combine technology above with pain relieving neurostimulator device such as TENS or PENS.

Aim to use pain relieving function prior to exercises to allow faster rehabilitation and reduce requirement for oral analgesia such as opiates.

Pain relieving device can be monitored in conjunction with the sensors to check if the use of a pain device speeds recovery.

Pain device can either delivered via two (in this embodiment) pads placed at the point of pain or via a wearable device placed across the joint.

Pain relief could, for example be provided using one or more of: neurostimulators and TENS, IFC and NMES. An example of a pain relief protocol could, for example, be based on the neurostimulation process

One aim may be to have either:
Monitoring sensors integrated into a wearable pain-relieving device (this would require sensors to work on all for joints where the pain device is used); or
Monitoring range of motion after the pain relief device has been used.

Associated software may present the same information with the following data:
Power level of pain relief delivered for each rehabilitation session to see how this relates to movement
Ability to check if rehab recovery times differ between the pads or wearable device.
Collection of data that can be used to show how (if) reducing pain with the device allows for not only quicker recovery but reduction in time pain relief is required.

Joints that may be monitored by devices/systems/methods formed in accordance with the present invention include, for example: hand joints; elbow joints; wrist joints; axillary articulations; sternoclavicular joints; vertebral articulations; temporomandibular joints; sacroiliac joints; hip joints; knee joints; and articulations of feet.

Sensor Technology

Each sensor module is entirely stand-alone and does not act like a conventional goniometer which relies upon the difference signal between one side of a joint and the other in order to obtain an angle. Instead, each sensor module is continuously reporting its orientation about its own internal tri-axial frame.

A significant contribution to this absolute reporting is the magnetometer component of the sensor which makes use of the earth's magnetic field in order to help determine the orientation of the sensor relative to the surface of the planet.

Therefore, by positioning one sensor at a known initial orientation we can then subsequently report all changes in angular orientation of this sensor relative to the initial orientation without reference to any other sensor.

Although only one sensor is required to measure multiplanar movement at a single joint, the use of two sensors either side of a joint, for example the knee, would allow monitoring of the entire limb segment simultaneously e.g. both hip and knee movement, as well as movement of the femur and tibia relative to the pelvis, could all be visualised simultaneously.

Uniquely for a single sensor, it is not absolutely necessary to align the axes of the sensor with the axes of rotation of the limb segment—any misalignment can be compensated for within a mathematical model. However better alignment means that any errors in the mathematical model will make a correspondingly smaller impact on the final measurement of angular orientation.

Uniquely therefore if one axis of the sensor is aligned parallel to the long axis of the limb segment then the sensor will report at all times the rotation angle of the limb segment about its long axis.

For this reason, as the anatomical features on one side of a joint are stationery throughout the examination—such as at the opposite shoulder or hip joint, then it is only necessary to deploy a single sensor on the upper arm or the thigh in order to obtain the angular displacement of the upper arm relative to the line through the shoulder-blades or to obtain the angular displacement of the thigh relative to the line through the pelvis.

As described in the previous point, when only a single sensor is required for measuring angles at the knee, hip or shoulder and elbow the sensor is placed either at the upper arm for the shoulder and elbow or thigh for the knee and hip. The mathematical model associated with the sensor compensates for slight changes in the positioning of the sensor meaning it doesn't need to be placed in exactly the same place every time.

The sensors can either be placed in a strapping around the arm or thigh (the nature of this strapping isn't limited and can be decided upon at the point of use or the preference of the patient/clinician.

The use of a single sensor to monitor multi-planar movements is unique and of commercial benefit as the cost per case is consequently reduced. The ability to use identical sensors and strapping for measurement of all the major joints is also unique.

In the embodiment shown the sensor is a combination of tri-axial accelerometer, tri-axial goniometer and tri-axial magnetometer. It employs firmware to interpret the raw signals from these constituent components in order to derive angular orientation data for the sensor about each of three mutually orthogonal axes. This orientation data is determined relative to an arbitrary cartesian coordinate frame of reference so that at any time the orientation of the sensor may be defined as lying parallel to any particular axis of the frame of reference (and thereby having an angular offset of zero) such that all subsequent measurements of orientation are defined relative to that reference orientation. This is important because it means that the sensor may be fitted to a limb in any orientation, and the wearer may be instructed to adopt any reference posture for that limb, and the sensor may then be ‘zeroed’ to that postural position, such that all subsequent measurements are defined relative to that reference postural position for the limb.

The model that interprets the raw signals may be an algorithm. It may be supplied as an accessory to the IMD component to support its use for converting raw signals into angular orientation.

The mathematical model for converting IMD output into meaningful limb and joint angles may be based on simple vector arithmetic.

Fundamentally the importance of the IMD based sensor of embodiments of the present invention is that it can report changes in orientation of a limb segment from a starting posture without reference to any other limb segment. This means that if a limb is moved relative to other non-moving anatomical parts, then the angle formed by that limb segment and the non-moving anatomical parts may be reported as a simple function of the single sensor. Specifically if the line between the shoulder blades has not moved and only the upper-arm has moved relative to that line between the shoulder blades—then it is sufficient to track the changes in orientation of the upper arm in order to report changes in angle between the upper arm and the line between the shoulder blades.

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention as defined by the appended claims and their equivalents.

1. A wearable sensor for providing real-time joint range of motion data, the sensor is stand-alone whereby only one sensor is needed to measure multiplanar movement at a single joint, the sensor comprises a combination of a tri-axial accelerometer, a tri-axial goniometer and a tri-axial magnetometer, the sensor continuously reports its orientation about its own internal tri-axial frame, and the sensor comprises firmware to interpret raw signals from these constituent components in order to derive angular orientation data for the sensor about each of three mutually orthogonal axes.

2-7. (canceled)

8. A sensor according to claim 1, in combination with a brace, strap or the like.

9. A sensor according to claim 1, in combination with a pain relieving device.

10. A sensor according to claim 9, in which the pain relieving device comprises a neurostimulator.

11-16. (canceled)

17. A patient rehabilitation system to provide real-time joint motion data to assess the capability of a joint, the system comprises a wearable sensor for providing real-time joint range of motion data, the sensor is stand-alone whereby only one sensor is needed to measure multiplanar movement at a single joint, the sensor comprises a combination of a tri-axial accelerometer, a tri-axial goniometer and a tri-axial magnetometer, the sensor continuously reports its orientation about its own internal tri-axial frame, and the sensor comprises firmware to interpret raw signals from these constituent components in order to derive angular orientation data for the sensor about each of three mutually orthogonal axes, the system comprises associated software, in which data is collected and transmitted from the sensor in real time and a visual demonstration of range of movement achieved is provided, and in which precise locational data provided by the sensor together with the associated software allows for an exact replication ofjoint movement to be shown to the patient to ensure compliance with a rehabilitation schedule.

18. A system according to claim 17, in which the sensor is provided in a removable strap or brace.

19. A system according to claim 17, further comprising means for administering pain relief.

20. A system according to claim 19, comprising a pain relieving neurostimulator device such as TENS or PENS.

21. A system according to claim 17, and configured for measurement of single plane motion including flexion and extension, and abduction/adduction movements and rotations movements.

22. A system according to claim 17, in which instructions can be sent and/or updated in real time by a physician or healthcare professional.

23. A system according to claim 17, in which a visual demonstration of range achieved is shown to a patient contemporaneously on a display screen.

24. A system according to claim 17, in which an algorithm is provided to interpret raw signals from the sensor, and in which the model that converts IMD output into meaningful limb and joint angles is based on simple vector arithmetic.

25. A system according to claim 17, in which orientation data is determined relative to an arbitrary cartesian coordinate frame of reference so that at any time the orientation of the sensor may be defined as lying parallel to any particular axis of the frame of reference, and thereby having an angular offset of zero, such that all subsequent measurements of orientation are defined relative to that reference orientation, whereby the sensor can be fitted to a limb in any orientation and the wearer can be instructed to adopt any reference posture for that limb, and the sensor may then be zeroed to that postural position, such that all subsequent measurements are defined relative to that reference postural position for the limb.

26. A system according to claim 17, configured to report changes in orientation of a limb segment from a starting posture without reference to any other limb segment, whereby if a limb is moved relative to other non-moving anatomical parts, then the angle formed by that limb segment and the non-moving anatomical parts may be reported as a simple function of the single sensor.

27. A system according to claim 17, being remotely monitorable by a clinician.

28. A system according to claim 17, comprising an alarm process if there are any concerning qualitative or qualitative deviations in terms of patient compliance and progress.

29. A system according to claim 17, connectable to an application on a mobile phone or tablet.

30. A method to provide real-time joint range of motion data comprising use of a standalone sensor positioned in an area of a subject to monitor that area and/or to monitor a site distant/remote from a physical sensor location, the method comprising the step of positioning a sensor at a known initial orientation and then subsequently reporting changes in angular orientation of the sensor relative to the initial orientation without reference to any other sensor, the sensor comprises a combination of a tri-axial accelerometer, a tri-axial goniometer and a tri-axial magnetometer, the sensor continuously reports its orientation about its own internal tri-axial frame, and the sensor comprises firmware to interpret raw signals from these constituent components in order to derive angular orientation data for the sensor about each of three mutually orthogonal axes.

31. A method according to claim 30, further comprising the step of administering pain relief before, during or after range of motion monitoring

32. A method according to claim 31, further comprising the step of determining if the use of pain relief speeds recovery
